(19) United States
(12) Patent Application Publication
(10) Pub. No.: US 2004/0247640 A1
(43) Pub. Date: Dec. 9, 2004

(54) Nitric oxide releasing eptfe coated medical device sandwich

(76) Inventors: Yi-Ju Zhao, Ellicott City, MD (US); William Herzog, Baltimore, MD (US)

Correspondence Address: Venable, PO Box 34385, Washington, DC, 20043-9998, US

(21) Appl. No.: 10/484,782

(22) PCT Filed: Jul. 23, 2002
(86) PCT No.: PCT/US02/23250

Related U.S. Application Data
(60) Provisional application No. 60/306,852, filed on Jul. 23, 2001.
(60) Provisional application No. 60/327,782, filed on Oct. 10, 2001.

(51) Int. Cl. A61F 2/00

(52) U.S. Cl. 424/423

(57) ABSTRACT

A device for contacting biological fluids in use that includes an intermediate layer between a first and a second preformed layer. The intermediate layer includes a permeating bioactive substance such as a drug or a pro-drug. At least one of the first or second layers is permeable to the drug or pro-drug to allow release into the biological fluid.

### BACKGROUND OF THE INVENTION

1. Field of the Invention

This invention relates to devices for use in applications involving contact with biological fluids. Specifically, the invention is a device for contacting biological fluids that releases a drug or pro-drug into the biological fluid.

2. Background

Invasive surgery and life saving techniques such as vascular grafting and dialysis require the contact or insertion of medical devices with or into biological fluids. The presence of such foreign material in the human body may result in several deleterious effects. In particular, when using devices such as vascular grafts and dialysis tubing, thrombogenesis may result in the blocking of the very blood flow that the devices are intended to facilitate. One solution to the thrombogenicity of devices inserted into biological fluids is through the systemic administration of anti-coagulant drugs such as warfarin, heparin, aspirin, clopidigrel, and ticlopidine. However, systemic administration of such drugs has several well recognized disadvantages, e.g. requirement the for long-term use of drugs, bleeding, thrombocytopenia and low patient compatibility.

There is a widespread need for techniques that improve surface properties of devices that are intended for uses where at least one surface comes in contact with biological fluids, particularly blood. In particular, it is desirable to modify blood-contacting surfaces, e.g. to prevent platelet adhesion and aggregation and neutrophil activation, and to prevent infection, which can result in deleterious effects. By modifying blood-contact properties of such surfaces, one can reduce or eliminate the need for systemic anti-coagulation therapy, extend the life expectancy of long-term implanted blood-contacting devices such as vascular grafts and stents, and improve the performance of shorter-term interventional devices, such as urinary and vascular catheters.

Invasive therapy such as vascular catheterization can be complicated by local infection and induced sepsis, which usually causes the failure of the therapy and is often life-threatening. About 6%˜10% catheters used for long-term venous access become infected (Bernard R W, et al., “Subclavian vein catheterization: a prospective study. II. Infectious complications,” Ann Surg 173:191, 1971; Uldall P R, Joy C, Merchant N., “Further experience with a double-lumen subclavian cannula for hemodialysis, Trans Am Soc Artif Intern Organs 28:71, 1982).

The catheter can allow microorganisms to gain access directly into the patient's vascular system. Biomaterials may alter host humoral and cellular immune response. The relatively hydrophobic property of the biomaterial makes it easy for bacteria to adhere to its surface. Endoscopic catheters and instruments suffer similar problems. Efforts have been made to reduce catheter infection, such as modifying the biomaterial surface to diminish bacterial adhesion, and binding antibiotics to the surface of biomaterials. However, none of these has been successfully used in clinical practice, and administering antibiotics systemically is unsatisfactory. Catheter-induced infection still remains a problem to be solved.

Another common complication from the use of inserted devices or devices used for extracorporeal flow of biological fluids is platelet aggregation and thrombogenesis.

There are several known techniques which have been tried to reduce thrombogenicity of medical devices by surface modification or coating. Several types of heparin coatings (covalent and ionic) have been produced. Phosphorylcholine coatings, marketed by Biocompatibles, Ltd., and described in U.S. Pat. No. 5,658,561, are at a very early stage of development and have not been well demonstrated.

Another technique to prevent thrombogenesis is release of NO from polymer films containing nitroso-containing compounds. Espadas-Torre, C., et al., “Thromboresistant chemical sensors using combined nitric oxide release/ion sensing polymeric films,” J. Am. Chem. Soc., 1997, 119:2321-2322. Nitric oxide-containing compounds may be characterized into several groups. (1) N-nitroso compounds are stable and do not readily release NO absent hydrolysis. In addition, N-nitroso compounds present risks of carcinogenicity. (2) A variety of S-nitrosothiols are known to generate NO in vivo. (3) C-nitroso compounds tend to be stable and release NO at body temperature, as in Rosen et al., U.S. Pat. No. 5,665,077. (4) Nitrosyl-containing organometallic compounds are described in Rosen et al., U.S. Pat. No. 5,797,887. According to the latter patent, decomposition of a nitrosyl-containing organometallic compound, such as nitroprusside, into NO is restricted by a polymer coating with a small porosity that inhibits the diffusion of blood-borne reductants to the NO-releasing compound; yet this small porosity allows NO to diffuse through the polymer into the surrounding fluid. There is a need for matrices demonstrating enhanced release of NO.

Green, U.S. Pat. No. 5,944,444, describes release of NO from biodegradable polymer matrices containing nitrites in an acid environment. Green et al., U.S. Pat. No. 5,814,666, describes N-nitroso compounds (NONOates) that release NO with antimicrobial effect upon hydrolysis when injected or ingested. Polymer matrices containing porosigens taught in the prior art, e.g., Eury, et al., U.S. Pat. No. 5,605,696, designed to facilitate the release of the therapeutic drug from the polymer coating into the vasculature, are unsatisfactory for enhancing nitric oxide release from nitric oxide donors.

Nitroprusside (as in, for example, sodium nitroprusside or SNP) has drawbacks when administered systematically as a NO donor, including short biological half time and systemic effects. There is a need for techniques that would prolong SNP biological effects and limit SNP effects to a local area.

Folts et al., WO 95/07691, describes using S-nitroso and other NO adducts mixed with bovine serum albumin on blood-contacting surfaces to inhibit platelet deposition. Such compositions are not biostable and allow the NO adduct to leach into the blood.

The use of other polymer matrices comprising SNP for coating medical devices has been described. For example, U.S. Pat. No. 5,797,887 and related publication WO98/08482, which are incorporated herein by reference in their entirety, describes a system for modifying blood contact surfaces by coating the surface with a polymer matrix comprising a NO precursor, such as SNP, where the polymer matrix releases NO without releasing the NO precursor. PCT application PCT/US01/08806, based on U.S. Application Nos. 60/190,571 and 60/190,546, which are incorporated herein by reference in their entirety, describe a system for modifying blood contact surfaces by coating the surface with a polymer matrix comprising a reducible NO donor, such as SNP, and a reductant that reacts with the reducible NO donor. These coatings are useful for preventing thrombosis and controlling microbial growth on the surface of the device. In use, the coating releases NO without releasing the reducible NO donor or the reductant.

Like systemic administration of anti-coagulants, surface modification of devices presents disadvantages, for example, undesirable modification of surface properties of the device, changes of physical properties of the device, such as becoming more rigid, less expandable, and deleterious effects of the coating on the healing process for a graft or stent.

The present invention overcomes these and other problems in the prior art.

### SUMMARY OF THE INVENTION

In summary, the invention is a device for contacting a biological fluid in use that includes an intermediate layer between an inner preformed layer and an outer preformed layer. The inner and outer preformed layers can be tubes. Blood is an exemplary biological fluid. The intermediate layer includes a permeating bioactive substance such as a drug or a pro-drug. At least one of the inner or outer layers contacts the biological fluid during use and this fluid contacting layer is permeable to the permeating bioactive substance drug or pro-drug to allow release into the biological fluid. The non-biological fluid contacting layer can be less permeable to the permeating bioactive substance than the biological fluid contacting layer. A less permeable or hydrophobic polymer may be formed on a surface of the non-biological fluid contacting layer to preferentially direct permeation of the permeating bioactive substance through the biological fluid contacting layer. Devices prepared according to the invention include, for example, a stent, a catheter, an extracorporeal blood transporting device such as dialysis tubing, and an intercorporeal blood transporting device such as a vascular graft.

The permeable layer of the device, which contacts a biological fluid and may be the inner or the outer layer, can be, for example, expanded polytetrafluorethylene. The permeating bioactive substance may be any solid, liquid or gas capable of permeating the permeable layer and “leaching” into the biological fluid. The permeating bioactive substance can be, for example, a gas.

Permeating bioactive substances may be nitric oxide and compounds, ions or moieties that produce nitric oxide, for example nitroprusside. Sodium nitroprusside decomposes to produce nitric oxide and is present in the intermediate layer in exemplary embodiments. Alternatively, the intermediate layer may contain a nitroprusside and a reducing agent capable of reducing the sodium nitroprusside to produce nitric oxide which is then released from the intermediate layer, through the permeable layer and into the blood, and/or into surrounding tissue such as blood vessel walls.

Devices according to the invention may be prepared by providing an inner layer and an outer layer, at least one of which is permeable to the permeating bioactive substance contained in an intermediate layer between the inner layer an the outer layer. The intermediate layer containing the permeating bioactive substance may be formed on the outside of the inner layer or on the inside of the outer layer followed by insertion of the inner layer into the outer layer. To prevent sliding of the inner layer through the outer layer, the inner and outer layers may be annealed over at least a portion of the device. The intermediate layer may also contain a rigid scaffold to maintain the shape of the device, e.g. in a graft. The ends of the device may be further annealed or enclosed within a relatively non-permeable polymer.

Devices according to the invention may be used by inserting the device into a biological fluid or a patient. In some embodiments, the biological fluid, for example blood, is directed to flow through or around the device.

The invention provides a medical device comprising: a first preformed layer, a second preformed layer, and an intermediate layer between said outer layer and said inner layer comprising a permeating bioactive substance; wherein at least one of said preformed layers is permeable to said permeating bioactive substance when contacting biological fluids. The at least one permeable layer may comprise expanded polytetrafluorethylene. The device may be tubular, where the first preformed layer is an inner tubular layer, and the second preformed layer is an outer tubular layer. The preformed inner layer and preformed outer layer together may comprise a unitary preformed material, for example in the form of a tube.

Both of the preformed layers are permeable or only one of the preformed layers is permeable. Both may have equal permeability or one layer may have more permeability. The permeable layer may be an inner layer and/or an outer layer. The permeating bioactive substance may comprise a substance that releases or reacts to release a bioactive substance, such as sodium nitroprusside releasing nitric oxide. The intermediate layer may comprise a reductant capable of reducing sodium nitroprusside to produce nitric oxide.

The bioactive substance may comprise a gas. The intermediate layer may comprise a polymer.

The device may comprise or be a stent, a catheter, an extracorporeal blood transporting device, an intercorporeal blood transporting device, or a vascular graft.

The preformed layers may be annealed over at least a portion of the device. The annealing may be at one or more ends of the device. The device may comprise a hydrophobic polymer, for example between at least one of the preformed layers and the intermediate layer.

The intermediate layer may comprise a tubular structure formed by coils of a flat helix with gaps between the coils of the helix, and the first and second preformed layers sheathing the flat helix, the first preformed layer forming a surface interior to the tubular structure and the second preformed layer forming a surface exterior to the tubular structure. The first and second preformed layers may be a unitary tube of expanded polytetrafluorethylene.

A device for contacting biological fluids according to the invention comprises: a preformed inner layer, a preformed outer layer, and an intermediate layer comprising sodium nitroprusside in a polymer matrix between said outer layer and said inner layer; the inner layer being permeable to nitric oxide released by reaction or decomposition of sodium nitroprusside and impermeable to sodium nitroprusside and the outer layer being impermeable to nitric oxide and sodium nitroprusside. The inner layer may comprise expanded polytetrafluorethylene. The polymer matrix may comprise polyvinyl alcohol. The intermediate layer may further comprise a reductant. The device may be a graft, an intercorporeal blood transfer device or an extracorporeal blood transfer device.

Another device for contacting biological fluids according to the invention in use comprises: a preformed inner layer, a preformed outer layer, and an intermediate layer between said outer layer and said inner layer comprising sodium nitroprusside in a polymer matrix; the outer layer being permeable to nitric oxide released by reaction or decomposition of sodium nitroprusside and impermeable to sodium nitroprusside and the inner layer being impermeable to nitric oxide and sodium nitroprusside. The outer layer may comprise expanded polytetrafluorethylene. The polymer matrix may comprise polyvinyl alcohol. The intermediate layer may further comprise a reductant. The device may be a stent or a catheter.

A method for making a device for insertion into a biological fluid according to the invention comprises: providing a preformed inner layer, providing a preformed outer layer, and forming an intermediate layer between the inner layer and the outer layer comprising a permeating bioactive substance, at least one of said inner layer and said outer later being permeable to said permeating bioactive substance. The intermediate layer may be formed on the outside surface of the inner layer and the outer layer may be applied to the inner layer thus forming an intermediate layer between the inner and outer layers. The intermediate layer may be on the inside surface of the outer layer and the inner layer may be applied to the intermediate layer coated outer layer to form the intermediate layer between the inner and outer layers.

A method of using the device of the invention comprises contacting said device with a biological fluid and releasing the permeating bioactive substance, for example inserting said device into a patient. One of the preformed layers may be an inner layer and one of the preformed layers may be an outer layer, and the method may further comprise directing the flow of a biological fluid into the device within the inner layer.

A method for making a device for insertion into a biological fluid according to the invention comprises: providing a preformed tube; inserting into the tube an intermediate layer comprising a permeating bioactive substance, said preformed tube comprising a material permeable to said permeating bioactive substance. The inserting step may comprise injecting. The method may comprise inserting an intermediate layer comprising inserting a structure coated with said permeating bioactive substance. The tube may comprise at least one preformed sheet, and the method may comprise sealing said tube. The structure may comprise a stent. The intermediate layer may comprise a polymer. The permeating bioactive substance may comprise sodium nitroprusside and/or nitric oxide. The polymer may comprising silicone.

Further objectives and advantages will become apparent from a consideration of the description, drawings, and examples.

### BRIEF DESCRIPTION OF THE DRAWINGS

FIG. 1 is a generalized schematic view of a device according to the invention.

FIG. 2 is a schematic of an embodiment of the invention prepared from a single preformed structure.

FIG. 3 is an embodiment of the invention prepared from preformed sheets.

FIG. 4 shows accumulation of NO released from a graft sandwich over a period of three weeks.

FIG. 5 shows a device according to the invention having partially connected inner and outer layers.

FIG. 6 shows release of NO from an intermediate layer comprising SNP and a reducing agent.

FIG. 7 is an internal structure of an aSpire™ stent available from Vascular Architects.

FIG. 8 is the structure of FIG. 5 coated with an SNP containing silicon polymer.

FIG. 9 shows release of NO from a stent comprising an SNP containing intermediate layer covered with expanded polytetrafluoroethylene.

FIG. 10 shows NO. release from a coated stent over two months. Data are the mean±SE (n=10). The dotted box shows half-time.

### DETAILED DESCRIPTION OF THE PREFERRED EMBODIMENTS

In describing preferred embodiments of the present invention, specific terminology is employed for the sake of clarity. However, the invention is not intended to be limited to the specific terminology so selected. All references cited herein are incorporated by reference as if each had been individually incorporated.

One purpose of this invention is to prevent thrombosis and intimal hyperplasia on the surface of a graft or other device inserted in a biological fluid. Biological fluids include blood, urine, bile and other fluids. The invention is particularly useful when the biological fluid is blood. It will be evident to persons skilled in the art that the invention is also useful for delivery of drugs from medical devices, particularly for the prevention of localized deleterious effects associated with the presence of inserted medical devices in the body. These effects are particularly significant when the device remains inserted for extended periods of time. Examples of such devices include stents, vascular grafts and other intracorporeal tubings that transport blood or other biological fluids within the body, catheters and extracorporeal tubing used to transport biological fluids outside the body, for example, dialysis tubing used to transport blood. Other possible devices that are within the scope of the invention include bags and other containers for holding or transporting blood or other biological fluids.

The primary clinical concern with artificial grafts and similar devices such as those described above is failure of the graft or device. Failure occurs most commonly in one of two ways: primary thrombosis or gradual occlusion of the lumen by intimal hyperplasia followed by secondary thrombosis. Short-term (within the first six weeks) failure is almost always due to primary thrombosis. Later failure generally occurs by thrombosis superimposed on a narrowed lumen. A variety of methods have been tried to prevent graft failure, including systemic anti-thrombotic medicine and various modifications of device surface. Despite these efforts, failure rates can be high. For example, the incidence of early failure of synthetic Arterio-venous fistula grafts may be as high as 27% [Surgery, 104:681, 1988]. The patency rate of ePTFE femoral-popliteal grafts has been reported as low as 42% at three years and the patency rate of heparin bonded Dacron polyester grafts has been reported at only 55% at three years [J. Vascular Surg., 33:533-539, 2001]. High failure rates limit the usefulness of currently available devices.

Expanded polytetrafluorethylene (ePTFE or Gore-Tex®) is a relatively bio-inert and non-thrombogenic material and is a common material for use in vascular grafts because of its case of use and biocompatibility. However, if an ePTFE graft is coated with some other polymer, the surface may become more thrombogenic, even if the polymer contains an anti-thrombosis drug and may cause an inflammatory reaction.

The present invention describes medical devices containing a permeating bioactive substance such as drugs or pro-drugs that take advantage of the favorable properties of ePTFE. In particular, devices according to the invention, such as vascular grafts, utilize ePTFE as the blood contacting device and thus, for all practical purposes, there is no modification of the surface of the device. Other materials having bio-compatibility characteristics similar to ePTFE are also within the scope of the invention.

An ePTFE graft is porous and is permeable to gases. Furthermore, the pore size of ePTFE can be adjusted by varying the amount of stretching during manufacture. Thus, it is possible that molecules larger than gases, for example, simple drugs and small peptides, may also permeate through ePTFE while the flow of water through the ePTFE remains restricted. Thus, devices comprising ePTFE as a blood contacting surface can release a drug or prodrug from beneath the surface into a biological fluid while restricting flow of biological fluids or other aqueous solutions through or beyond the device. As used herein, a “permeating bioactive substance” refers to a substance that is bioactive and able to permeate ePTFE, such as a drug, or a substance that becomes biologically active upon exposure to the biological fluid, such as a prodrug and able to permeate ePTFE, or a compound that reacts within the device to produce a bioactive substance that can permeate ePTFE. A permeating bioactive substance and the bio-active substance itself may be a solid, liquid, gas or a solute in a solid, liquid or gas. Exemplary bioactive substances are gases. As applied here, permeation of the permeating bioactive substance means permeation of either the substance contained in the intermediate layer, i.e. the permeating bioactive substance itself, or a substance derived from the permeating bioactive substance, such as nitric oxide derived from SNP, where SNP is the permeating bioactive substance

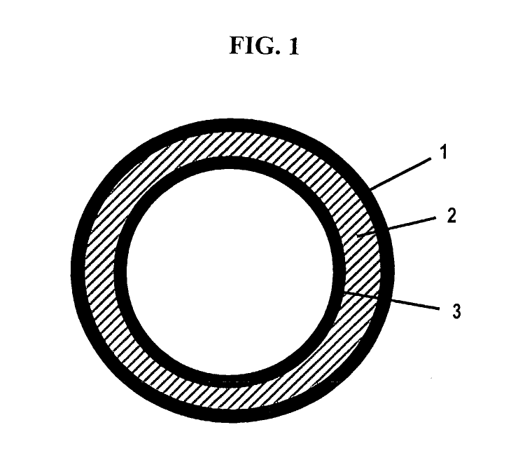

FIG. 1 is a generalized schematic view of an embodiment of a device according to the invention. The device includes three layers: an outer layer 1, an inner layer 3 and an intermediate “drug storage” layer 2 between the inner layer 3 and outer layer 1. The intermediate layer 2 includes a permeating bioactive substance. Depending on the use of the device, the inner layer 3, outer layer 1 or both may comprise of ePTFE. At least the layer which will contact a biological fluid into which the permeating bioactive substance will permeate can be comprised of, for example, ePTFE. The non-biological fluid contacting layer can be less permeable than ePTFE. The device thus is as an intermediate drug containing layer “sandwiched” between an inner layer and an outer layer. Alternatively, both the inner and outer layers may be permeable to the permeating bioactive substance.

While inner and outer are used above to identify two different preformed structures, inner and outer may also refer to the orientation of the device. Thus, inner and outer may be defined by the surface to which the device is directed, even if a single preformed structure is used to prepare the inner and outer layers. For example, an intermediate layer may be inserted into a preformed tube and the ends may be sealed. If the structure is then flattened and placed, for example, against the side of a vessel, the side of the structure facing the lumen of the vessel may be regarded as the inner layer, whereas the side of the device adjacent to the vessel wall may be regarded as the outer layer.

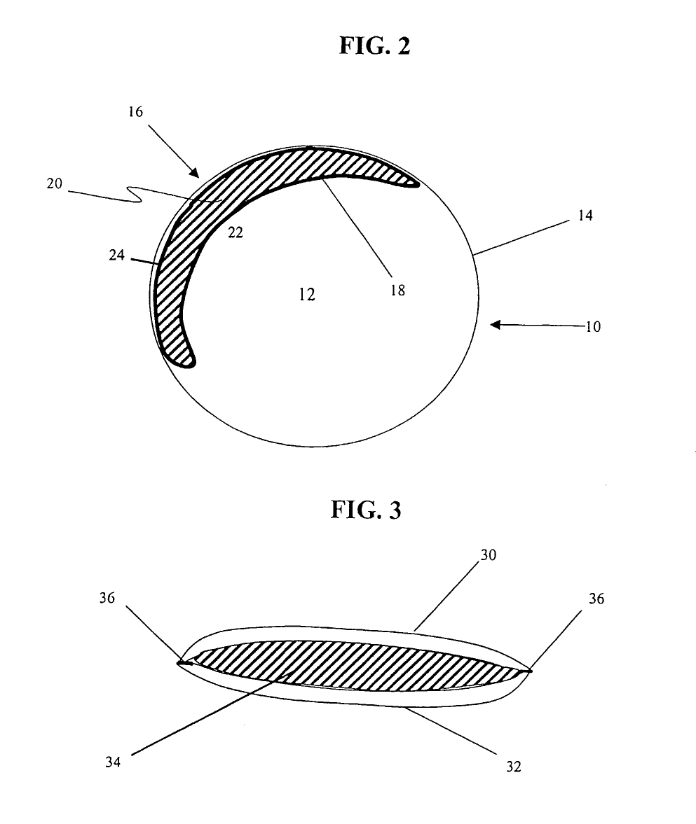

FIG. 2 is a schematic of an embodiment of the invention prepared from a single preformed structure in a vessel. The vessel 10 comprises a lumen 12 and a wall 14. The device 16 of the invention is shown in cross section. The wall 18 of the device 16 comprises a single preformed structure. The intermediate layer 20 comprises a permeating bio-substance. In this device, the surface 22 adjacent to the lumen 12 of the vessel 10 is the inner layer of the device. The surface 24 of the device adjacent to the wall 14 of the vessel 10 the outer layer of the device.

FIG. 3 is a cross section of an embodiment of the invention prepared from preformed sheets. The embodiment comprises a first preformed sheet 30, a second preformed sheet 32, and an intermediate layer 34. Depending on the orientation of the device in use, the first preformed sheet 30 may be the inner or outer layer of the device. Similarly, the second preformed sheet 32 may be the outer or inner layer. The preformed sheets of this embodiment may be annealed at the edges 36 where they meet.

The present invention differs from the prior art where multilayered structures are formed by sequentially placing multiple coatings on a preformed device, for example, a stent or a catheter. According to the present invention, the first and second layers, which may be an inner and an outer layer, are preformed. These preformed structures may be, for example, sheets or, in exemplary embodiments, tubes. The structures may be woven, extruded or prepared by any other means suitable to the particular application. The intermediate layer is fixed between the tubes as described hereinafter. Fixing the intermediate layer prevents it from leaking out of the two layers.

The bodily fluid-contacting layer may comprise, for example, ultra thin ePTFE, and such as an ePTFE graft. This layer functions to (a) separate the intermediate drug storage layer from blood; and (b) control drug release. The intermediate layer includes the permeating bioactive substance which may be, for example, a drug, a pro-drug or a substance that releases a drug or pro-drug, and may also include a polymer. This layer provides a drug source and may also include a release control element. The remaining layer may comprise a less permeable material. For example, the remaining layer may be made of thin walled ePTFE. To further decrease permeability, the internal surface of the non-biological fluid contacting layer, i.e. the surface adjacent to the intermediate layer, may be coated with a hydrophobic polymer. The hydrophobic polymer can be less permeable to the bioactive substance than the ePTFE layer to provide preferential permeation of the bioactive substance through the blood-contacting layer.

In embodiments where blood or some other biological fluid flows through the device, the inner layer 3 is permeable and comprises, for example, ultra thin walled ePTFE. The outer layer 1 can comprise a less permeable polymer such as thicker walled ePTFE or a hydrophobic polymer. When prepared in this way, the permeating bioactive substance in the intermediate layer 2 preferentially permeates a bioactive substance from the intermediate layer and into the interior of the tube, where the biological fluid is present. Thus, deleterious effects on the inside of the device are minimized. For example, by using an antithrombogenic permeating bioactive substance in a graft, thrombosis inside the graft is reduced. This embodiment of the invention is particularly useful for the preparation of devices that are intercorporeal tubings for the flow of blood, such as vascular grafts, and extracorporeal tubing for the flow of blood, such as dialysis tubing. This embodiment is also useful for devices that are designed to contain a biological fluid, for example, a bag for holding or transporting blood.

In embodiments where blood or other biological fluid flows around the device, the outer layer 1 is permeable and can comprise, for example, ultra thin walled ePTFE. The inner layer 3 can be prepared from a less permeable polymer such as thicker walled ePTFE or a hydrophobic polymer. When prepared in this way, the permeating bioactive substance of the intermediate layer 2 preferentially permeates from the intermediate layer to the exterior of the tube, where the biological fluid is present. Thus, deleterious effects on the outside of the device are minimized. For example, by using an antithrombogenic permeating bioactive substance on a catheter, thrombosis around the catheter is prevented. This embodiment of the invention is particularly useful for the preparation of devices such as catheters and stents. Notably the inner layer 3 may be a polymer coated directly on the device such as a catheter or stent.

Nitric oxide and compounds that react or decompose to produce nitric oxide are exemplary permeating bioactive substances. Although the permeating bioactive substance may be any drug, pro-drug or drug or pro-drug precursor capable of permeating through the ePTFE layer, nitric oxide is an exemplary drug. The production of nitric oxide in a polymer matrix coated on a device has been described, for example in U.S. Pat. No. 5,797,887 and related publication WO98/08482 and PCT application PCT/US01/08806, based on U.S. Application Nos. 60/190,571 and 60/190,546. Both of these PCT applications are incorporated herein by reference in their entirety. The intermediate layer of the present invention may be, for example, a matrix as described in these two previous examples. These previously prepared matrix systems may include reducing agents. Thus, as will be obvious to persons skilled in the art, the intermediate layer of the present invention may include other adjuvants that activate or react with the permeating bioactive substance prior to permeation from the intermediate layer and release from the device.

In general, devices according to the present invention may be prepared by coating a hydrophobic polymer on the inside surface of the outer layer or on the outside surface of the inner layer. The permeating bioactive substance is then deposited on the hydrophobic layer. The device is then assembled by inserting the inner layer into the outer layer. For example, an outer layer ePTFE tubing may be coated on the inside surface with silicon. A permeating bioactive substance, for example SNP, is deposited on the silicon. Deposition can occur before curing. A thin walled tubing of ultra thin ePTFE forming the inner layer is then inserted into the coated ePTFE.

Alternatively, the SNP may be combined with a polymer prior to coating. In this embodiment of the preparation process, a hydrophobic polymer such as silicon may be applied to the inside surface of the outer inner layer ePTFE tubing. The permeating bioactive substance, for example SNP, either alone or mixed with a hydrophilic polymer such as, for example polyvinyl alcohol (PVA), is applied to the outside surface of the inner layer. The permeating bioactive substance coated inner layer is then inserted into the hydrophobic polymer coated outer layer.

In other embodiments, it may be possible to insert the inner layer into the outer layer before forming the intermediate layer. The intermediate layer can then be inserted by, for example, injecting the permeating bioactive substance or a polymer matrix containing the permeating bioactive substance between the layers. One disadvantage to this method is that it may be more difficult to form an intermediate layer having a uniform thickness throughout the periphery of the inner layer.

While the above manufacturing process has been outlined for embodiments having the inner layer as the biological fluid contacting surface, analogous procedures may be used to prepare devices where the outer layer is intended to be the biological fluid contacting layer. In addition, other components may be present in the intermediate layer, such as components that add structural integrity. For example, in a stent graft or similar device, the stent portion may be included in the intermediate layer.

Embodiments of the invention prepared from a single preformed structure may be prepared in several ways. First, the intermediate layer may be injected into a preformed tube. The intermediate layer of this embodiment comprises a permeating bioactive substance and may also include a polymer matrix, and/or a reductant or other additive to enhance or retard release of the permeating bioactive substance. When prepared according to this embodiment, the ends of the preformed tube may be sealed.

Alternatively, embodiments prepared from a single preformed structure may be prepared by depositing the permeating bioactive substance on a structure that becomes part of the intermediate layer. The permeating bioactive substance may be dispersed in a polymer matrix before being applied to the structure. The coated structure may then be inserted into the preformed structure, such as an ePTFE tube. The ends of the tube may then be sealed.

Embodiments of the invention prepared from preformed sheets may also be prepared in various ways. In a first method, the permeating bioactive substance, optionally in a polymer matrix and optionally containing other additives as described herein, is applied to a first preformed sheet to form on intermediate layer. A second preformed sheet is placed on the permeating bio-substance containing intermediate layer. The first and second sheets may then be annealed at the edges to form the device.

Alternatively, two preformed sheets may be annealed along two or three sides to form a sealed or open tube. Devices according to the invention can then be prepared as with preformed tubes by, for example, injecting the intermediate layer or placing a coated structure in the sealed or open tube.

Bonding some polymers to ePTFE and ePTFE may be difficult and result in “slipping” of the inner layer through outer layer. Specifically, if the intermediate layer does not bond well to ePTFE, the layer formed from ePTFE may slide through the outer layer resulting in a loss of structural integrity. Accordingly, in some embodiments of the invention it may be necessary to anneal portions of the inner and outer layer or otherwise fix the intermediate layer between the inner and outer layers. FIG. 5 shows an example of a device 4 annealed according to this method. The annealed layers of the device have annealed portions 5 arranged in a regular pattern. This annealing essentially “quilts together” the inner and outer layers so that slippage does not occur. Of course, such annealing need not be at regular intervals. Another means for improving the bonding is to incorporate a more “sticky” polymer into the intermediate layer.

To prevent leaching of the permeating bioactive substance from the intermediate layer, it may be necessary to seal the ends of the device, eliminating exposure of the intermediate layer to the environment. This may be accomplished by, for example, applying a polymer or other sealant that is impermeable to the permeating bioactive substance to the ends of the device. For example, a hydrophobic polymer such as silicon may be applied at the ends. Alternatively, the ends of the device may be sealed by a continuous annealing of the inner layer to the outerlayer.

### EXAMPLE 1

A ePTFE graft sandwich consisting of an inner layer of ultra thin walled ePTFE (I.D. 3 mm) and an outer layer of thin walled ePTFE (I.D. 3.5 mm). The interior surface of the outer layer was coated with silicone. Sodium nitroprusside powder was applied to the coating surface and the coating cured. The 3 mm ePTFE was placed in the silicon and SNP coated 3.5 mm graft.

An experimental apparatus consisting of a pump, a 15 ml reservoir, PVC tubing and the graft sandwich (7 cm long) was constructed, forming a closed loop. The graft sandwich was connected in the middle of PVC tubing. The system was filled with phosphate buffered saline (38 ml) and a flow rate of 40 ml/min established while shielding the graft segment from light and maintaining room temperature. Samples were collected periodically. After sample collection, perfusate was replaced with fresh buffer. Nitric oxide (NO) concentration (the permeated bioactive substance) of the perfusate was measured by using Griess reagents.

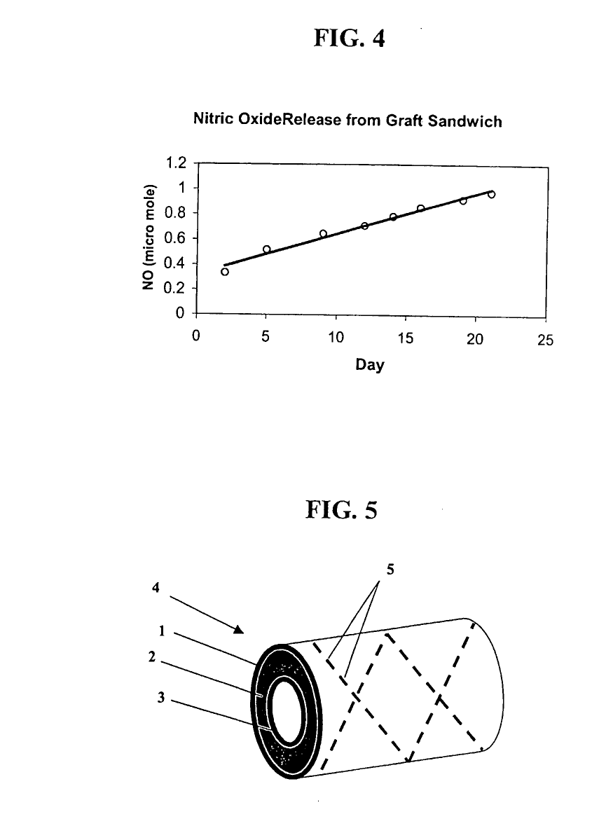

FIG. 4 shows a steady accumulation of NO released from the graft sandwich over three weeks. This establishes that the device of the invention can release a drug over an extended period.

### EXAMPLE 2

An ePTFE graft sandwich was prepared having an outer layer of ePTFE graft (I.D. 4 mm, regular wall, 7 cm), and an inner layer of ePTFE (I.D. 3 mm, thin wall, 9 cm). The inside surface of the outer layer was coated with silicone. Before the silicone coating was cured, SNP powder was applied on the surface of the coating. The outer surface of the inner layer was coated with 5% PVA containing 10% L-ascorbic acid. Ascorbic is a reducing agent that reacts with SNP to release NO. After the coatings cured, the inner layer was inserted into the outer layer. Both ends were sutured and sealed with silicone.

The graft sandwich was connected with a reservoir by PVC tubing as in Example 1. The system was filled with 30 ml phosphate buffered saline, and driven by a rotation pump. Flow rate was 40 m/min. NO released from the graft sandwich was evaluated by use of Griess reagents.

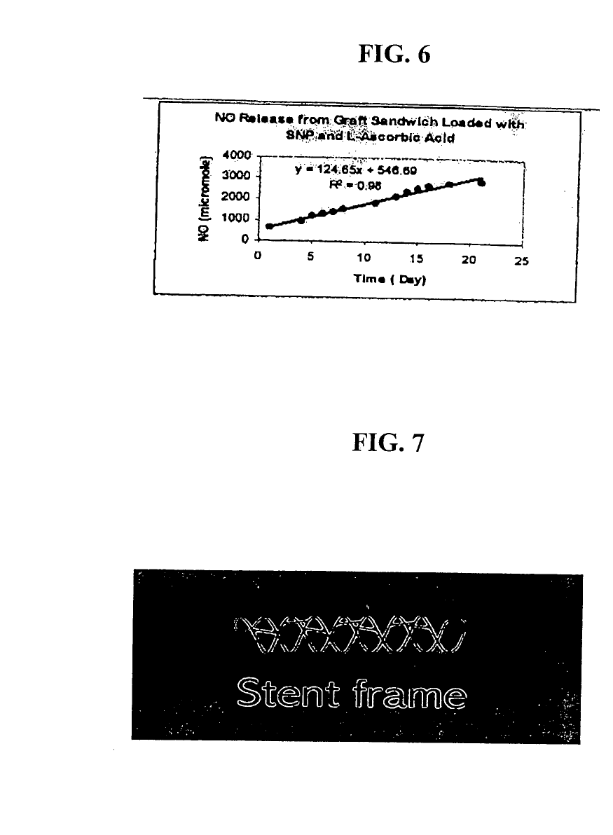

FIG. 6 shows a steady release of NO from the graft sandwich, demonstrating NO release in the presence of a reducing agent over a three week period.

### EXAMPLE 3

An NO releasing stent was prepared using an aSpire™ Stent from Vascular Architects. The coating of the stent was removed to reveal the underlying metallic framework. A 10% SNP/silicone solution was prepared by adding 1 gm fine SNP powder (Sigma, Lot#119H2481) into 10 ml silicone (Rhodia, High strength silicone, Lot#21849, Solids content: 40%), and mixing. Coatings were prepared by two methods:

Method 1—The metallic framework of the stent was inserted into an ePTFE tube and one end sealed. The SNP/Silicone solution was injected into the ePTFE tube. After the silicone was cured, the other end of the ePTFE tube was sealed. A thin film is formed inside the tube.

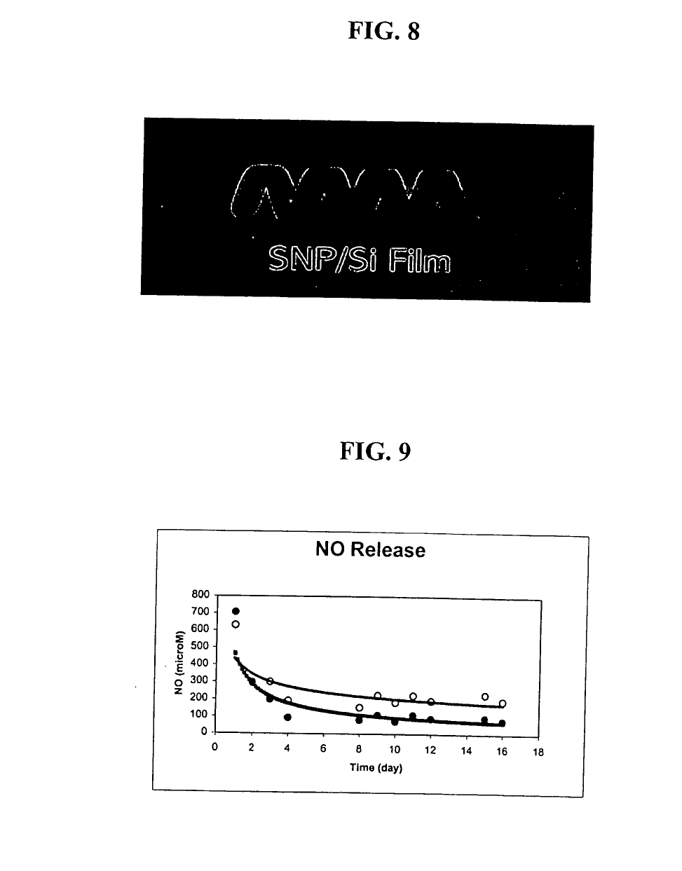

Method 2—The metal frame of the stent was stretched (FIG. 7). The SNP/silicone solution was cast onto the open space in the metal frame. After the silicone cured and ePTFE tube dried, the metal frame with SNP/silicone film (FIG. 8) was placed inside the ePTFE tube. Both ends of the ePTFE tubes were then sealed.

Six stents prepared using method 1 and three stents prepared using method 2 were tested for NO release. One uncoated stent was used as a control. Test tubes each containing one covered stent and 4 ml phosphate buffered saline (PBS) were placed in a 37° C. incubator. Samples were collected from the PBS buffer in the test tubes and the buffer was replaced with fresh PBS after sample collection. NO concentration was measured by use of Griess reaction. NO release over a sixteen day period is shown in FIG. 9. The upper line (open data points) represents Method 1, and the lower line (solid data points) represents Method 2. FIG. 9 shows a steady release of NO from the device over a period greater than two weeks.

### EXAMPLE 4

To effectively inhibit platelet function and intimal hyperplasia, and so prevent restenosis after implantation of a stent, the medical device needs to release NO. over an extended period in the human circulatory system.

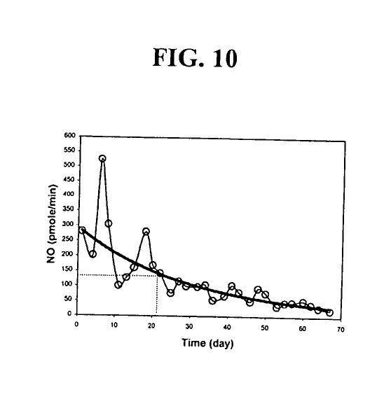

The interior of an aSpire® VA stent was coated with silicone into which SNP was incorporated (30% by weight). An in vitro experiment was conducted with ten coated stent-grafts and two uncoated controls in a flow system for 67 days. The objective was to determine how much NO. would be released and for how long in a room temperature system circulating 5 mL buffer solution at 100 mL/min. Samples were taken each Monday, Wednesday, and Friday and measured using the Griess reaction. Afterwards the buffer was replaced. The results demonstrate that the coated device releases NO. over two months (FIG. 10).

The NO. release curve peaks during the first week, and then decreases slowly over the experimental period. The higher initial levels of NO. release can inhibit thrombosis in the short term, and the lower subsequent levels can inhibit hyperplasia over the longer term. An in vivo porcine animal study confirmed the efficacy of the SNP-silicone coating in preventing restenosis in the carotid artery 28 days after stenting.

The positive data with the covered stent-model contrasts with the negative results from the experiments in Yoon et al, Yonsei Medical Journal, vol. 43, No. 2, pp. 242-251 (2002). The inventive stent is superior probably due to the choice of polymer and the ability to incorporate more NO-donor onto a graft-covered stent versus an uncovered stent.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, and elements added or omitted, without departing from the invention, as appreciated by those skilled in the art in light of the above teachings. It is therefore to be understood that, within the scope of the paragraphs and their equivalents, the invention may be practiced otherwise than as specifically described.

1. A medical device comprising:
a first preformed layer,
a second preformed layer, and
an intermediate layer between said outer layer and said inner layer comprising a permeating bioactive substance;
wherein at least one of said preformed layers is permeable to said permeating bioactive substance when contacting biological fluids.

2. The device of claim 1, the at least one permeable layer comprising expanded polytetrafluorethylene.

3. The device of claim 1, wherein the device is tubular, the first preformed layer is an inner tubular layer, and the second preformed layer is an outer tubular layer.

4. The device of claim 1, the preformed inner layer and preformed outer layer together comprising a unitary preformed material.

5. The device of claim 4, the unitary preformed material comprising a tube.

6. The device of claim 1, wherein both of the preformed layers are permeable.

7. The device of claim 1, wherein only one of the preformed layers is permeable.

8. The device of claim 3, wherein the permeable layer is the inner layer.

9. The device of claim 3, wherein the permeable layer is the outer layer.

10. The device of claim 3, wherein the device comprises a stent.

11. The device of claim 1, wherein the permeating bioactive substance comprises a substance that releases or reacts to release a bioactive substance.

12. The device of claim 1, wherein the bioactive substance is nitric oxide.

13. The device of claim 1, the permeating bioactive substance comprising sodium nitroprusside.

14. The device of claim 13, the intermediate layer further comprising a reductant capable of reducing sodium nitroprusside to produce nitric oxide.

15. The device of claim 1, the bioactive substance comprising a gas.

16. The device of claim 1, the intermediate layer further comprising a polymer.

17. The device of claim 1, selected from the group of a stent, a catheter, an extracorporeal blood transporting device, an intercorporeal blood transporting device, and a vascular graft.

18. The device claim 1, wherein said preformed layers are annealed over at least a portion of the device.

19. The device of claim 18, where the annealing is at the end of the device.

20. The device of claim 1, further comprising a hydrophobic polymer.

21. The device of claim 1, comprising a hydrophobic polymer between at least one of the preformed layers and the intermediate layer.

22. The device of claim 1, the intermediate layer comprising a tubular structure formed by coils of a flat helix with gaps between the coils of the helix, and the first and second preformed layers sheathing the flat helix, the first preformed layer forming a surface interior to the tubular structure and the second preformed layer forming a surface exterior to the tubular structure.

23. The device of claim 22, the first and second preformed layers being a unitary tube of expanded polytetrafluorethylene.

24. A device for contacting biological fluids in use comprising:
a preformed inner layer,
a preformed outer layer, and
an intermediate layer comprising sodium nitroprusside in a polymer matrix between said outer layer and said inner layer;
the inner layer being permeable to nitric oxide released by reaction or decomposition of sodium nitroprusside and impermeable to sodium nitroprusside and the outer layer being impermeable to nitric oxide and sodium nitroprusside.

25. The device of claim 24, the inner layer comprising expanded polytetrafluorethylene.

26. The device of claim 24, the polymer matrix comprising polyvinyl alcohol.

27. The device of claim 24, the intermediate layer further comprising a reductant.

28. The device of claim 24, said device being a graft, an intercorporeal blood transfer device or an extracorporeal blood transfer device.

29. A device for contacting biological fluids in use comprising:
a preformed inner layer,
a preformed outer layer, and
an intermediate layer between said outer layer and said inner layer comprising sodium nitroprusside in a polymer matrix;
the outer layer being permeable to nitric oxide released by reaction or decomposition of sodium nitroprusside and impermeable to sodium nitroprusside and the inner layer being impermeable to nitric oxide and sodium nitroprusside.

30. The device of claim 29, the outer layer comprising expanded polytetrafluorethylene.

31. The device of claim 29, the polymer matrix comprising polyvinyl alcohol.

32. The device of claim 29, the intermediate layer further comprising a reductant.

33. The device of claim 29, said device being a stent or a catheter.

34. A method for making a device for insertion into a biological fluid comprising:
providing a preformed inner layer,
providing a preformed outer layer,
forming an intermediate layer between the inner layer and the outer layer comprising a permeating bioactive substance;
at least one of said inner layer and said outer later being permeable to said permeating bioactive substance.

35. The method of claim 34, comprising forming the intermediate layer on the outside surface of the inner layer and then applying the outer layer to the inner layer thus forming an intermediate layer between the inner and outer layers.

36. The method of claim 34, comprising forming the intermediate layer on the inside surface of the outer layer and then applying the inner layer to the outer layer to form the intermediate layer between the inner and outer layers.

37. A method of using the device of claim 1, comprising contacting said device with a biological fluid and releasing the permeating bioactive substance.

38. A method of using the device of claim 1, comprising inserting said device into a patient

39. The method of claim 37, wherein one of the preformed layers is an inner layer and one of the preformed layers is an outer layer, and further comprising directing the flow of a biological fluid into the device within the inner layer.

40. A method for making a device for insertion into a biological fluid comprising:
providing a preformed tube;
inserting into the tube an intermediate layer comprising a permeating bioactive substance;
said preformed tube comprising a material permeable to said permeating bioactive substance.

41. The method of claim 40, said inserting comprising injecting.

42. The method of claim 40, said inserting an intermediate layer comprising inserting a structure coated with said permeating bioactive substance.

43. The method of claim 40, said tube comprising at least one preformed sheet.

44. The method of claim 40, further comprising sealing said tube.

45. The method of claim 42, said structure comprising a stent.

46. The method of claim 40, said intermediate layer further comprising a polymer.

47. The method of claim 40, said permeating bioactive substance comprising sodium nitroprusside.

48. The method of claim 46, said polymer comprising silicone.
